SEQUENCE LISTING (2 sequences)

SEQ ID NO: 1 - DNA, 31 bp, Artificial Sequence
  Synthetic Construct
cttcttggatccgatgaccactctgtgccgc

SEQ ID NO: 2 - DNA, 30 bp, Artificial Sequence
  Synthetic Construct
cttcttaagctttcattaatcggcggtctg

(19) United States
(12) Patent Application Publication
(10) Pub. No.: US 2021/0009939 A1
(43) Pub. Date: Jan. 14, 2021

(54) COMPOSITIONS AND METHODS FOR MAXIMIZING MALONYL-COA IN E. COLI

(71) Applicant: BOARD OF SUPERVISORS OF LOUISIANA STATE UNIVERSITY AND AGRICULTURAL AND MECHAN, Baton Rouge, LA (US)

(72) Inventors: Grover L. Waldrop, Baton Rouge, LA (US); Tatiana Thompson Silveira Mello, Baton Rouge, LA (US); Marcio de Queiroz, Baton Rouge, LA (US)

(21) Appl. No.: 16/922,493

(22) Filed: Jul. 7, 2020

Related U.S. Application Data
(60) Provisional application No. 62/871,328, filed on Jul. 8, 2019.

(51) Int. Cl. C12N 1/20

(52) U.S. Cl. CPC C12N 1/20; C12N 2523/00; C12N 2500/16; C12N 2500/34; C12N 2500/14; C12N 2500/74

(57) ABSTRACT

Disclosed herein is a low-cost method to maximize malonyl-CoA production in E. coli, and consequently a high yield of its derived bioproducts.

### CROSS-REFERENCE TO RELATED APPLICATIONS

This application claims benefit of U.S. Provisional Application No. 62/871,328, filed Jul. 8, 2019, which is hereby incorporated herein by reference in its entirety.

### SEQUENCE LISTING

This application contains a sequence listing filed in electronic form as an ASCII.txt file entitled “221205-1340 Sequence Listing_ST25” created on Jul. 6, 2020. The content of the sequence listing is incorporated herein in its entirety.

### BACKGROUND

As the world's population steadily increases, there is a concomitant increase in the demand for energy. The current petrochemical industry controls most of the production of transportation energy and other petrochemicals. The instability of petroleum prices, the limited availability, and its impact in the environment make it necessary to look for alternative feedstocks that can sustain the profitability of energy companies. To this end, industrial biotechnology uses microorganisms and enzymes to produce a wide range of chemical compounds.

The three-carbon metabolite malonyl-CoA can serve as a precursor to a variety of industrial chemicals. Malonyl-CoA is the product of the reaction catalyzed by acetyl-CoA carboxylase (ACC), which is the first committed and regulated step in fatty acid biosynthesis (Broussard, et al. 2013. Biochemistry, 52(19): 3346-3357). The reaction catalyzed by ACC involves two half-reactions and is shown in FIG. 1. Bacterial ACC is composed of three proteins: biotin carboxylase (BC), carboxyltransferase (CT), and biotin carboxyl carrier protein (BCCP). In the first half reaction, BC catalyzes an ATP-dependent carboxylation of biotin, which is covalently attached to BCCP. In the second half reaction, CT transfers the carboxyl group from biotin to acetyl-CoA to produce malonyl-CoA. Enzymatic activity requires all three of these proteins to form a macromolecular complex, hereafter referred to as holo ACC (FIG. 1) (Broussard, et al. 2013. Biochemistry, 52(19): 3346-3357).

The major hurdle with using malonyl-CoA as a precursor in biotechnology processes is that the intracellular concentration is low, 35 μM (Zhao, et al. 2009. Metabolic Engineering, 11(3): 192-198). The low concentration stems from the fact that holo ACC does not catalyze the reverse or non-physiological reaction, and therefore, the reaction is not at equilibrium (Broussard, et al. 2013. Biochemistry, 52(19): 3346-3357). As a consequence, the levels of malonyl-CoA are low, while the levels of the substrate acetyl-CoA, which determines the activity of holo ACC (Broussard, et al. 2013. Biochemistry, 52(19): 3346-3357) vary depending on the metabolic state of the cell. Previous attempts to increase the intracellular amount of malonyl-CoA have ranged from genetic engineering of proteins involved in fatty acid biosynthesis (Rathnasingh et al. 2009. Applied Microbiology and Biotechnology, 84(4): 649-657) to adding inhibitors of enzymes in fatty acid synthesis (U.S. Pat. No. 8,883,464). All of these approaches have led to modest increases in intracellular malonyl-CoA, often at great expense. In this invention, it is described a straightforward, low cost method to significantly increase the intracellular level of malonyl-CoA that can be broadly applied to the production of a number of industrial chemicals and bioproducts.

### SUMMARY

Disclosed herein are enhanced bacteria, systems, and methods that can be used to maximize malonyl-CoA production in bacterial culture The malonyl-CoA produced according to the disclosed methods can then eb converted into a chemical product of interest.

In some embodiments, the disclosed method for maximizing malonyl-CoA production in bacterial culture involves preparing a bacterial inoculum by culturing a bacterial colony in a first bacterial medium at about 30-39° C. for 19 to 24 hours under aerobic conditions, and then culturing the inoculum at about 0.5-2% (v/v) in a second bacterial medium. Therefore, in some embodiments, the bacterial colony is cultured in the first bacterial medium at about 30-39° C., 30-35° C., 36-39° C., or 32-37° C. for about 12 to 48 hours, including about 12 to 24, 12 to 36, 19 to 24, 19 to 36, 24 to 36, or 24 to 48 hours under aerobic conditions. In some embodiments, the inoculum is cultured in about 0.5-2%, 0.5-1%, or 1-2% (v/v) in a second bacterial medium.

In some embodiments, the first bacterial medium and second bacterial medium are identical. In some embodiments, the first and second bacterial medium are different, in order to stimulate different metabolic pathways. For example, in some embodiments the first bacterial medium is a rich medium and the second is a minimal medium.

In some embodiments, the first or second bacterial medium is produced from purified water supplemented with from 0 to 0.5 mg/L magnesium, from 0 to 0.1 mg/L manganese, from 0 to 6 mg/L calcium, or any combination thereof. For example, in some embodiments, the first or second bacterial medium is produced from purified water supplemented with from 0 to 0.2, 0.3 to 0.5, or 0.1 to 0.4 mg/L magnesium, including about 0.01, 0.02, 0.03, 0.04, 0.05, 0.10, 0.15, 0.20, 0.25, 0.30, 0.35, 0.40, 0.45, or 0.50 mg/L magnesium. In some embodiments, the first or second bacterial medium is produced from purified water supplemented with from 0 to 0.05, 0.06 to 0.10, or 0.3 to 0.08 mg/L manganese, including 0.005, 0.01, 0.02, 0.03, 0.04, 0.05, 0.06, 0.07, 0.08, 0.09, or 0.10 mg/L manganese. In some embodiments, the first or second bacterial medium is produced from purified water supplemented with from 0 to 3, 4 to 6, or 2 to 4 mg/L calcium, including about 0.1, 0.2, 0.3, 0.4, 0.5, 0.6, 0.7, 0.8, 0.9, 1.0, 2.0, 3.0, 4.0, 5.0, or 6.0 mg/L calcium

In some embodiments, the bacterial medium contains from 5 to 1000 mM glucose, including about 5 to 100, 10 to 200, or 50 to 150 mM glucose, such as 5, 6, 7, 8, 9, 10, 11, 12, 13, 14, 15, 16, 17, 18, 19, 20, 25, 30, 35, 40, 45, 50, 60, 70, 80, 90, 100, 110, 120, 130, 140, 150, 160, 170, 180, 190, 200, 300, 400, 500, 600, 700, 800, 900, or 1000 mM glucose. In some embodiments, the bacterial medium does not contain glucose.

In some embodiments, the bacterial medium has a pH of from 6.0 to 7.5, including about 6.0 to 7.0, 6.5 to 7.0, 6.5 to 7.5, or 7.0 to 7.5, such as 6.0, 6.1, 6.2, 6.3, 6.4, 6.5, 6.6, 6.7, 6.8, 6.9, 7.0, 7.1, 7.2, 7.3, 7.4, or 7.5.

In some embodiments, the bacterial medium contains from 0 to 35 mg/ml lactose, including about 0 to 15, 0 to 20, 0 to 30, 5 to 15, 5 to 20, 5 to 30, 5 to 35, 10 to 15, 10 to 20, 10 to 30, 10 to 35, 15 to 20, 15 to 30, 15 to 35, 20 to 25, 20 to 30, 20 to 35, 25 to 30, 25 to 35, or 30 to 35 mg/ml lactose, such as 0, 1, 2, 3, 4, 5, 6, 7, 8, 9, 10, 11, 12, 13, 14, 15, 16, 17, 18, 19, 20, 25, 30, or 35 mg/ml lactose.

In some embodiments, the bacterial medium contains LB, TB, 2XYT, or any combination of yeast extract and tryptone, and MOPS, or M9 minimal medium. In some embodiments, the bacterial medium does not contain IPTG.

In some embodiments, the aerobic conditions involve a concentration of O₂ of from 0% to 25%, including about 0% to 5%, 0% to 10%, 0% to 15%, 0% to 20%, 0% to 25%, 5% to 10%, 5% to 15%, 5% to 20%, 5% to 25%, 10% to 15%, 10% to 20%, or 15% to 25%, such as 0.01%, 0.05%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9. %8, 9.9%, 10%, 11%, 12%, 13%, 14%, 15%, 16%, 17%, 18%, 19%, 20%, 20.1%, 20.2%, 20.3%, 20.4%, 20.5%, 20.6%, 20.7%, 20.8%, 20.9%, 21%, 22%, 23%, 24%, or 25%.

In some embodiments, the aerobic conditions involve from 0.04 to 25% concentration of CO₂, including about 0.04 to 1%, 0.04 to 10%, 0.04 to 15%, 0.04 to 20%, 1 to 10%, 15 to 20%, 10 to 25%, or 1 to 25%.

In some embodiments, the bacteria used in the disclosed methods is a thermophilic or a mesophilic bacterium. In certain embodiments, the thermophilic or mesophilic bacterium is a species of the genera Escherichia, Propionibacterium, Thermoanaerobacterium, Thermoanaerobacter, Clostridium, Geobacillus, Saccharococcus, Paenibacillus, Bacillus, Caldicellulosiruptor, Anaerocellum, Anoxybacillus, Klebsiella, Lactobacillus, Lactococcus, or Corynebacterium. In some embodiments, the bacterial colony is E. coli.

In some embodiments, the bacterial colony is recombinantly engineered to overexpress malonyl-CoA. In some embodiments, the bacterial colony is recombinantly engineered to overexpress acetyl-CoA carboxylase and its subunits. In some embodiments, the bacterial colony is recombinantly engineered to overexpress 1,3,6,8-tetrahydroxynaphtalene synthase. In some embodiments, the bacterial colony is not recombinantly engineered.

In some embodiments, the method further involves product extraction. For example, if the final product is secreted out of the cell, then cells can be discarded, whereas if the product remains in the cytosol then the cells can be frozen.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 shows the reactions of biotin carboxylase (BC) and carboxyltransferase (CT) along with the sum of the entire acetyl-CoA carboxylase reaction.

FIG. 2 illustrates fermentation steps utilized to maximize intracellular malonyl-CoA according to some embodiments of the disclosure.

FIG. 3 shows the reaction catalyzed by 1,3,6,8-tetrahydroxynaphtalene synthase (THNS).

FIG. 4 is a bar graph showing effects of type of media in the production of flaviolin. The control group was not induced with lactose, while the second one indicates use of 250 mg.

FIG. 5 is a plot showing carbon source effect on the flaviolin production varying with six concentrations (0, 0.1 mM, 1 mM, 10 mM, 100 mM and 1M).

FIG. 6 is a plot showing production of flaviolin affected due the supplementation of varying amounts of glucose in the inoculums which were used to inoculate the cultures.

FIG. 7 is a bar graph showing minimal medium effect on the flaviolin production.

FIG. 8 is a bar graph showing richer medium effect on the flaviolin production.

FIG. 9 is a bar graph showing effect of metal supplementations on the production of flaviolin.

FIG. 10 is a bar graph showing inducer effect (Lactose vs IPTG) on the production of flaviolin.

FIG. 11 is a bar graph showing flaviolin production when inducing the cultures just after inoculation (t_induction=0 hour), and when [(OD)]_600=0.3 t_induction=2 hours). Blue bars had the temperature kept at 37° C. during the induction (T_ind) and the growth (T_gwt). Orange bars had the inducer added at T_ind=25° C., after that the temperature was shifted to T_gwt=37° C. Yellow bars had the inducer added at T_ind=37° C., after that the temperature was shifted to T_gwt=25° C. Purple bars had the temperature kept at 25° C. during induction and growth.

FIG. 12 is a bar graph showing temperature influence on the production of flaviolin.

FIG. 13 is a graph showing effect of time of incubation of the inoculum on the production of flaviolin.

FIG. 14 is a bar graph showing effect of the types of closure on the flaviolin production.

FIG. 15 is a bar graph showing the effect of overexpression of holo ACC (pAEP7+pSEB1) and holo ACC with biotin ligase (pAER1+pLB0056) comparing with only pAER1.

FIG. 16 is a bar graph showing the effect of CO2 on the production of flaviolin.

FIG. 17 illustrates products produced from malonyl-CoA.

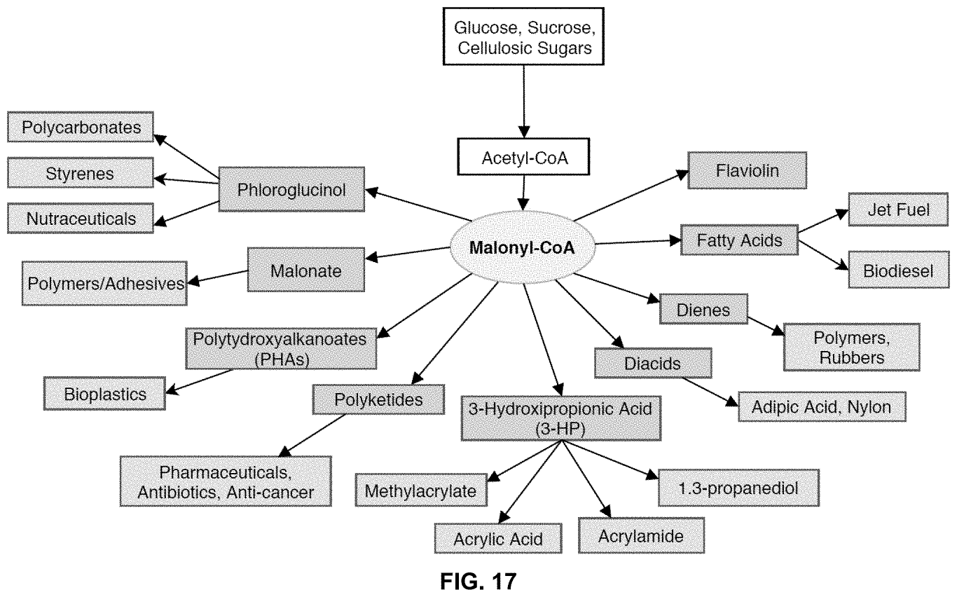

FIG. 18 illustrates some variables for malonyl-CoA maximization.

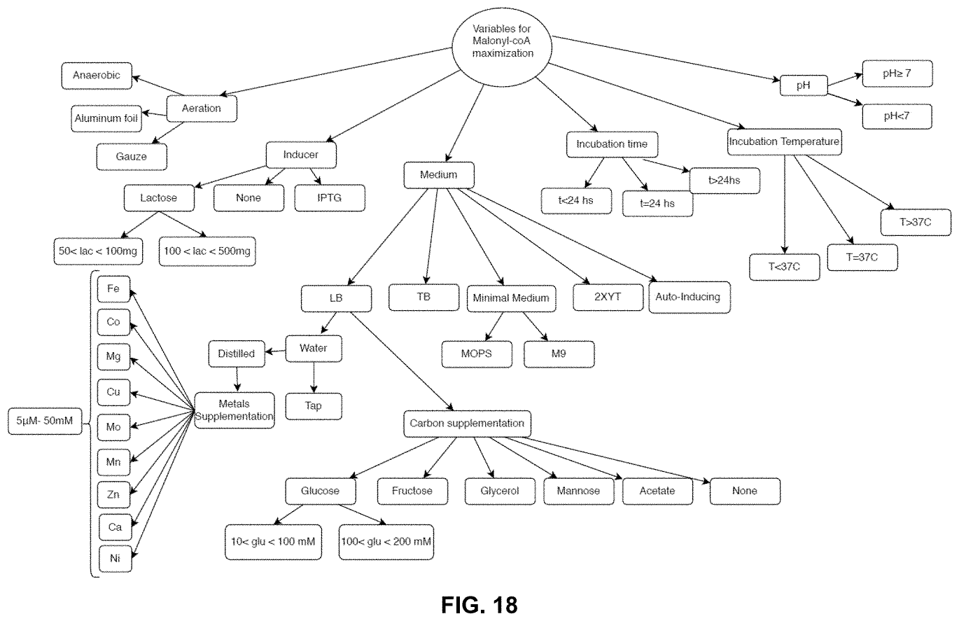

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

As used herein, the term “malonyl-CoA derived product” or “malonyl-CoA derived bioproduct” is intended to include those products that are synthesized from, derived from, or are used as an intermediate in their synthesis from malonyl-CoA. The term includes products such as hydrocarbons, hydrocarbon derivatives, polyketides, organic acids, including but not limited to adipic acid and 3-hydroxyproprionate, and any other products from which malonyl-CoA can serve as a precursor.

In some embodiments, the bacteria used in the disclosed methods is a thermophilic or a mesophilic bacterium. In certain embodiments, the thermophilic or mesophilic bacterium is a species of the genera Escherichia, Propionibacterium, Thermoanaerobacterium, Thermoanaerobacter, Clostridium, Geobacillus, Saccharococcus, Paenibacillus, Bacillus, Caldicellulosiruptor, Anaerocellum, Anoxybacillus, Klebsiella, Lactobacillus, Lactococcus, or Corynebacterium. In other embodiments, the microorganism is a bacterium selected from the group consisting of: E. coli strain B, strain C, strain K, strain W, Shewanella, Propionibacterium acnes, Propionibacterium freudenreichii, Propionibacterium shermanii, Propionibacterium pentosaceum, Propionibacterium arabinosum, Clostridium acetobutylicum, Clostridium beijerinckii, Thermoanaerobacterium thermosu/furigenes, Thermoanaerobacterium aotearoense, Thermoanaerobacterium polysaccharolyticum, Thermoanaerobacterium zeae, Thermoanaerobacterium xylanolyticum, Thermoanaerobacterium saccharolyticum, Thermoanaerobium brockii, Thermoanaerobacterium thermosaccharolyticum, Thermoanaerobacter thermohydrosu/furicus, Thermoanaerobacter ethanolicus, Thermoanaerobacter brocki, Clostridium thermocellum, Clostridium clariflavum, Clostridium cellulolyticum, Clostridium phytofermentans, Clostridium straminosolvens, Geobacillus thermog/ucosidasius, Geobacillus stearothermophilus, Saccharococcus caldoxylosilyticus, Saccharoccus thermophilus, Paenibacillus campinasensis, Bacillus flavothermus, Anoxybacillus kamchatkensis, Anoxybacillus gonensis, Caldicellulosiruptor acetigenus, Caldicellulosiruptor saccharolyticus, Caldicellulosiruptor kristjanssonii, Caldicellulosiruptor owensensis, Caldicellulosiruptor lactoaceticus, Lactobacillus thermophilus, Lactobacillus bulgaricus, Lactococcus lactis, and Anaerocellum thermophilum. In one embodiment, recombinant microorganism is selected from the group consisting of Clostridium thermocellum, and Thermoanaerobacterium saccharolyticum.

Suitable bacterial media for used in the disclosed methods include commercially prepared media such as Luria Bertani (LB) broth, M9 minimal media, Sabouraud Dextrose (SD) broth, Yeast medium (YM) broth, and yeast synthetic minimal media (Ymin). Other defined or synthetic bacterial media may also be used, and the appropriate medium for growth of the particular microorganism will be known by one skilled in the art of microbiology or bio-production science. In various embodiments, a minimal media may be developed and used that does not comprise, or that has a low level of addition of various components, for example less than 10, 5, 2 or 1 g/L of a complex nitrogen source including but not limited to yeast extract, peptone, tryptone, soy flour, corn steep liquor, or casein. These minimal media may also have limited supplementation of vitamin mixtures including biotin, vitamin B12 and derivatives of vitamin B12, thiamin, pantothenate and other vitamins. Minimal media may also have limited simple inorganic nutrient sources containing less than 28, 17, or 2.5 mM phosphate, less than 25 or 4 mM sulfate, and less than 130 or 50 mM total nitrogen. Minimal media may also have limited supplementation of trace metals including manganese, boron, cobalt, copper, molybdenum, zinc, calcium, magnesium, iron, and nickel.

Any of the enhanced bacteria as described may be introduced into an industrial bio-production system where the enhanced bacteria convert a carbon source into a selected chemical product in a commercially viable operation. The bio-production system includes the introduction of enhanced bacteria into a bioreactor vessel, with a carbon source substrate and bio-production media suitable for growing the enhanced bacteria, and maintaining the bio-production system within a suitable temperature range (and dissolved oxygen concentration range if the reaction is aerobic or microaerobic) for a suitable time to obtain a desired conversion of a portion of the substrate molecules to the chemical product. Industrial bio-production systems and their operation are well-known to those skilled in the arts of chemical engineering and bioprocess engineering.

Bio-productions may be performed under aerobic, microaerobic, or anaerobic conditions, with or without agitation. The operation of cultures and populations of microorganisms to achieve aerobic, microaerobic and anaerobic conditions are known in the art, and dissolved oxygen levels of a liquid culture comprising a nutrient media and such microorganism populations may be monitored to maintain or confirm a desired aerobic, microaerobic or anaerobic condition. When syngas is used as a feedstock, aerobic, microaerobic, or anaerobic conditions may be utilized. When sugars are used, anaerobic, aerobic or microaerobic conditions can be implemented in various embodiments.

Any of the enhanced bacteria as described may be introduced into an industrial bio-production system where the microorganisms convert a carbon source into chemical products in a commercially viable operation. The bio-production system includes the introduction of such a recombinant microorganism into a bioreactor vessel, with a carbon source substrate and bio-production media suitable for growing the enhanced bacteria, and maintaining the bio-production system within a suitable temperature range (and dissolved oxygen concentration range if the reaction is aerobic or microaerobic) for a suitable time to obtain a desired conversion of a portion of the substrate molecules to the chemical product.

A classical batch bioreactor system is considered “closed” meaning that the composition of the medium is established at the beginning of a respective bio-production event and not subject to artificial alterations and additions during the time period ending substantially with the end of the bio-production event. Thus, at the beginning of the bio-production event the medium is inoculated with the desired organism or organisms, and bio-production is permitted to occur without adding anything to the system. Typically, however, a “batch” type of bio-production event is batch with respect to the addition of carbon source and attempts are often made at controlling factors such as pH and oxygen concentration. A variation on the standard batch system is the fed-batch system. Fed-batch bio-production processes comprise a typical batch system with the exception that the nutrients, including the substrate, are added in increments as the bio-production progresses. Fed-Batch systems are useful when catabolite repression is apt to inhibit the metabolism of the cells and where it is desirable to have limited amounts of substrate in the media. Measurement of the actual nutrient concentration in Fed-Batch systems may be measured directly, such as by sample analysis at different times, or estimated on the basis of the changes of measurable factors such as pH, dissolved oxygen and the partial pressure of waste gases such as CO2. Batch and fed-batch approaches are common and well known in the art and examples may be found in Thomas D. Brock in Biotechnology: A Textbook of Industrial Microbiology, Second Edition (1989) Sinauer Associates, Inc., Sunderland, Mass., Deshpande, Mukund V., Appl. Biochem. Biotechnol., 36:227, (1992), and Biochemical Engineering Fundamentals, 2′d Ed. J. E. Bailey and D. F. 011 is, McGraw Hill, New York, 1986, herein incorporated by reference for general instruction on bio-production.

In some embodiments, the disclosed enhanced bacteria are used in continuous bio-production methods. Continuous bio-production is considered an “open” system where a defined bio-production medium is added continuously to a bioreactor and an equal amount of conditioned media is removed simultaneously for processing. Continuous bio-production generally maintains the cultures within a controlled density range where cells are primarily in log phase growth. Two types of continuous bioreactor operation include a chemostat, wherein fresh media is fed to the vessel while simultaneously removing an equal rate of the vessel contents. The limitation of this approach is that cells are lost and high cell density generally is not achievable. In fact, typically one can obtain much higher cell density with a fed-batch process. Another continuous bioreactor utilizes perfusion culture, which is similar to the chemostat approach except that the stream that is removed from the vessel is subjected to a separation technique which recycles viable cells back to the vessel. This type of continuous bioreactor operation has been shown to yield significantly higher cell densities than fed-batch and can be operated continuously. Continuous bio-production is particularly advantageous for industrial operations because it has less down time associated with draining, cleaning and preparing the equipment for the next bio-production event. Furthermore, it is typically more economical to continuously operate downstream unit operations, such as distillation, than to run them in batch mode.

Continuous bio-production allows for the modulation of one factor or any number of factors that affect cell growth or end product concentration. For example, one method will maintain a limiting nutrient such as the carbon source or nitrogen level at a fixed rate and allow all other parameters to moderate. In other systems a number of factors affecting growth can be altered continuously while the cell concentration, measured by media turbidity, is kept constant. Methods of modulating nutrients and growth factors for continuous bio-production processes as well as techniques for maximizing the rate of product formation are well known in the art of industrial microbiology and a variety of methods are detailed by Brock, supra.

Also disclosed herein is a system for bio-production of a chemical product as described herein, said system comprising: a fermentation tank suitable for cell culture of the disclosed enhanced bacteria; a line for discharging contents from the fermentation tank to an extraction and/or separation vessel; and an extraction and/or separation vessel suitable for removal of the chemical product from cell culture waste. In various embodiments, the system includes one or more pre-fermentation tanks, distillation columns, centrifuge vessels, back extraction columns, mixing vessels, or combinations thereof.

The following published resources are incorporated by reference herein for their respective teachings to indicate the level of skill in these relevant arts, and as needed to support a disclosure that teaches how to make and use methods of industrial bio-production of 3-HP, or other product(s) produced under the invention, from sugar sources, and also industrial systems that may be used to achieve such conversion with any of the recombinant microorganisms of the present invention (Biochemical Engineering Fundamentals, 2nd Ed. J. E. Bailey and D. F. 011is, McGraw Hill, New York, 1986, entire book for purposes indicated and Chapter 9, pages 533-657 in particular for biological reactor design; Unit Operations of Chemical Engineering, 5th Ed., W. L. McCabe et al., McGraw Hill, New York 1993, entire book for purposes indicated, and particularly for process and separation technologies analyses; Equilibrium Staged Separations, P. C. Wankat, Prentice Hall, Englewood Cliffs, N.J. USA, 1988, entire book for separation technologies teachings). Generally, it is appreciated, in view of the disclosure, that any of the above methods and systems may be used for production of various chemical products such as those disclosed herein.

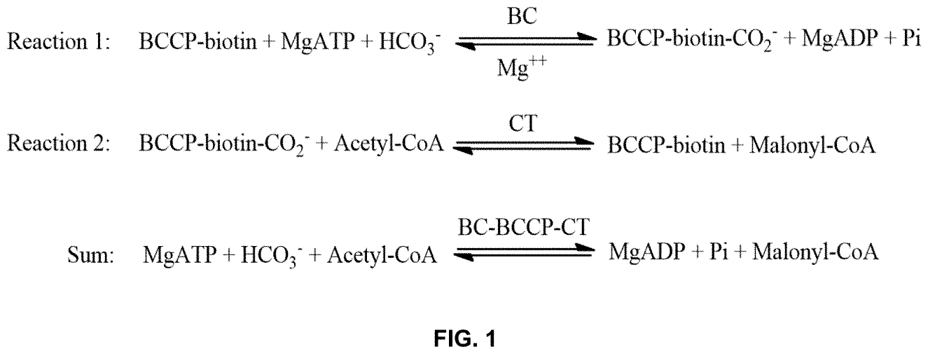

Disclosed herein are enhanced bacteria, systems, and methods that can be used to convert malonyl-CoA to a chemical product of interest. Numerous products can be made from malonyl-coA precursors alone by expressing enzyme functions to convert malonyl-coA into products. Several examples of these non-limiting products are shown below in FIG. 1. Hexaketide pyrone can be made by expressing a hexaketide pyrone synthase from either Aloe arborescens or Plumbago indica. Octaketide 4b pyrone can be made by expressing an octaketide 4b pyrone synthase from Aloe arborescens. Octaketide can be made by expressing an octaketide synthase from Hypericum perforatum. Pentaketide chromone can be made by expressing a pentaketide chromone synthase from Aloe arborescens. 3-hydroxypropionic acid can be made by expressing a malonyl-coA reductase and 3-hydroxypropionic acid dehydrogenase from various sources.

One chemical product is 3-hydroxypropionic acid (CAS No. 503-66-2, “3-HP”). Chemical products further include tetracycline, erythromycin, avermectin, macrolides, vanomycin-group antibiotics, Type II polyketides, (5R)-carbapenem, 6-methoxymellein, acridone, actinorhodin, aloesone, apigenin, barbaloin, biochanin A, maackiain, medicarpin, cannabinoid, cohumulone, daidzein, flavonoid, formononetin, genistein, humulone, hyperforin, mycolate, olivetol, pelargonidin, pentaketide chromone, pinobanksin, pinosylvin, plumbagin, raspberry ketone, resveratrol, rifamycin B, salvianin, shisonin, sorgoleone, stearate, anthocyanin, ternatin, tetrahydroxyxanthone, usnate, and xanthohumol. Particular polyketide chemical products include 1,3,6,8-tetrahydroxynaphthalene (THN) or its derivative flaviolin (CAS No. 479-05-0). The production of 3-HP, or of THN or flaviolin, may be used herein to demonstrate the features of the invention as they may be applied to other chemical products. Alternatively, any of the above compounds may be excluded from a group of chemical products.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1: Development of a Low-Cost Method for Maximizing Malonyl-CoA in E. coli

A good strategy is to inhibit the fatty acid synthesis in E. coli since it would not only eliminate a competing pathway consuming malonyl-CoA, but also alleviate the inherent negative regulation. Several studies have attempted to use metabolic engineering to decrease the fatty acids synthesis in recombinant E. coli strains, in order to improve the carbon flux towards acetyl and malonyl-CoA. Using this approach, Lynch et al achieved the highest titer reported thus far of 3-Hydroxypropionic acid (U.S. Pat. No. 8,883,464). Lu et al introduced four distinct genetic changes into the E. coli genome, in which one of them was to overexpress ACC to metabolic engineer an efficient producer of fatty acids which is used to synthesize microbial biodiesel (Lu, et al. 2008. Metabolic Engineering 10(6):333-39). Cells treated with cerulenin or thiolactomycin, had malonyl-CoA as the dominant component, since these are antibiotics that inhibit β-keto-acyl ACP synthase and acetyl-CoA ACP transacylase of fatty acid synthase (Zhang, et al. 2006. J Biol Chem 281(26):17541-44). Acetyl-CoA carboxylase is well known to be the first enzyme of the biosynthetic sequence of fatty acid synthesis, but it is possible that this enzyme is not the crucial pacemaker of fatty acids synthesis. Therefore, the goal is to understand how nutrients and physiological conditions play a role into the optimization of ACC, and maximization of malonyl-CoA in E. coli.

The first step in optimizing ACC activity in the cell is being able to measure its activity in vivo. Most previous studies utilize sophisticated and complex methods to quantify intermediate compounds, such as, High Performance Liquid Chromatography (HPLC), isotopic labeling, mass spectrometry, and enzymatic-specific assays (Zha, et al. 2009. Metabolic Engineering 11(3):192-98; Fowler, et al. 2009. Appl Environ Microbiol 75(18):5831-39; Rathnasingh, et al. 2012. J Biotechnol 157(4):633-40; WO2012129450A1; Atsumi, et al. 2008. Metabolic Engineering 10(6):305-11). All these high cost methods go against the requirement of developing an economically viable process. Therefore, the intracellular malonyl-CoA is quantified using the enzyme 1,3,6,8-tetrahydroxynapthalenesynthase (THNS). THNS catalyzes condensation of five molecules of malonyl coenzyme A (CoA) to form 1,3,6,8-tetrahydroxynaphthalene (THN). THN is readily converted into 2,5,7-trihydroxy-1,4-naphthoquinone (flaviolin) by auto-oxidation and secreted out of the cell (Zhao et al. 2005). Flaviolin is randomly polymerized to form red-brown compound which protects the hosts against ultraviolet (UV) radiation (Miyahisa, et al. 2005. Appl Microbiol Biotechnol. 68(4):498-504). Therefore, the in vivo quantification of malonyl-CoA can be directly done measuring the absorbance of flaviolin with a spectrophotometer, turning the quantitative data acquisition fast and economically feasible. Therefore, a low-cost method was developed to maximize and quantify malonyl-CoA production in E. coli, and consequently a high yield of its derived bioproducts.

### Materials and Methods

The E. coli strain BL21(DE3) and the expression vector pCDFDuet-1 were from Novagen. Restriction enzymes, dNTPs, and T4 DNA ligase were from New England Biolabs. Primers were purchased from MWG Biotech. Isopropyl β-D-1-thiogalactopyranoside (IPTG) was from Gold Biotechnology. In addition, the plasmid pLB0056, which contains the genes for holo ACC and biotin ligase, was provided. All other reagents were from Sigma.

The gene for 1,3,6,8-tetrahydroxynapthalene synthase (THNS) was provided. The THNS gene was amplified using the forward primer 5′-CTTCTTGGATCCGATGACCACTCTGTGCCGC-3′ (SEQ ID NO:1) and backward primer 5′-CTTCTTAAGCTTTCATTAATCGGCGGTCTG-3′ (SEQ ID NO:2). The PCR product was cut with BamHI and HindIII then inserted into pAEP9, which was cut with the same two restriction enzymes. This generated the plasmid pSEB1, containing not only the gene for THNS but also the genes for the α and β subunits of CT, which are cloned into a mini operon on pCDFDuet-1. The E. coli strain BL21(DE3) was transformed with pSEB1 and pAEP7, which contained the genes for BCCP and BC in a mini operon cloned into pET-28. Co-transformation of pSEB1 and pAEP7 was possible because pET28 and pCDFDuet-1 have different origins of replication. E. coli strain BL21(DE3) was transformed with the plasmid pAER1 which contains the amplified THNS gene. In order to evaluate the different subunits of ACC individually, the THNS gene from pSEB1 was subcloned into the BamHI and HindIII sites of pCDFDuet-1 to generate pAER1.

TABLE 1
List of all plasmids used in this work

| Plasmid | Description |
|---|---|
| pAER1 | THNS |
| pAEP3 | Holo BCCP |
| pAEP7 | BCCP and BC |
| pAEP9 | α and β of CT |
| pSEB1 | pAEP9 and THNS in pCDFDuet-1 |
| PLBOO56 | Holo ACC and Biotin Ligase |

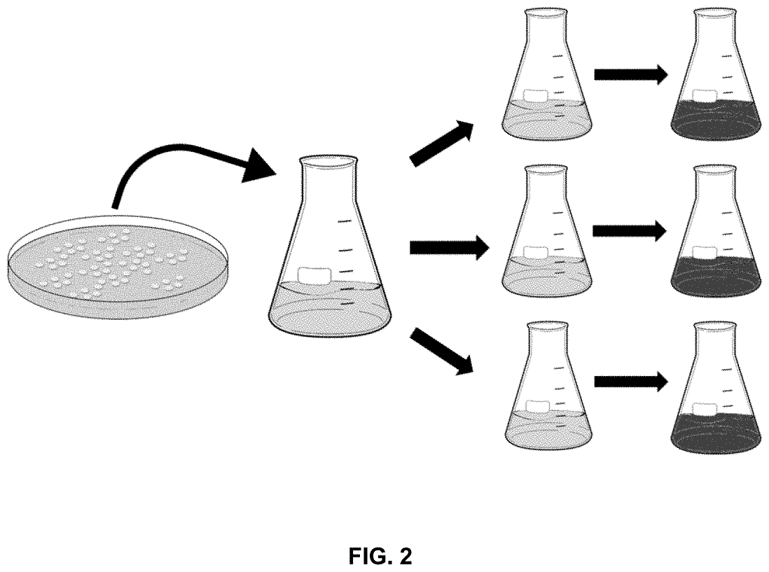

Unless otherwise stated, the following three phases for the in vivo assay was the standard procedure used for all experiments (FIG. 2). Plate: Luria Bertani agar plates are streaked colony from the permanent and incubated for 20 hours at 37° C. Inoculum: 10 ml of medium was added to 125 mL flask and supplement with carbon sources. A single colony from LB agar plate was used to inoculate the flasks, and 30 μL of antibiotics 50 mg/ml was added. Flasks were covered with aluminum foil, making holes to increase aeration. Incubation was done overnight in a shaking water bath at 37° C. and 250 rpm. Cultures: 5 ml of autoclaved medium was added to a 125 ml flask, 1% (v/v) of the inoculum was transferred and gene overexpression was induced with lactose or IPTG. Reading: 1 mL sample of the culture was centrifuged for 150 seconds at 13,500 g, and 250 μL of the supernatant was added to 750 μL of water (1:4 dilution). The absorbance was measured at 340 nm (0D340, 1 cm path length) using a Cary 60 UV-Vis spectrophotometer from Agilent Technologies. The blank standard was a 1:4 dilution of the medium. The concentration of flaviolin was determined using the extinction coefficient ϵ=3,068 M⁻¹ cm⁻¹(Krauser, et al. 2012. ChemCatChem 4(6):786-88). All experiments were done in triplicate. Results were reported as the concentration of flaviolin per gram wet-weight of bacterial cells in a 1 mL sample.

### Cultures and Growth Media

LBT (Luria Bertani Tap) rich medium used in the inoculum and cultures contained (per liter): 10 g tryptone, 5 g yeast extract, 5 g NaCl, and complete with tap water to 1000 ml.

LBM (Luria Bertani Modified) rich medium used in the inoculum and cultures contained (per liter): 10 g tryptone, 5 g yeast extract, 5 g NaCl, glucose and micronutrients solution as indicated, and complete with deionized (DI) water to 1000 ml.

2XYT rich medium (per liter): 16 g tryptone, 10 g yeast extract, 5 g NaCl, and complete with tap water to 1000 ml.

TB rich medium (per liter): 12 g tryptone, 24 g yeast extract, 4 ml glycerol, and complete with tap water to 1000 ml.

M9 minimal media (per liter): 780 ml sterile water, 200 ml 5×M9 salts, 2 mL of 1M MgSO₄, 0.1 mL 1M CaCl₂, 5 ml of thiamine hydrochloride (5 mg/m1), 1 mL 20% glucose, 3 ml of antibiotics, and complete with DI water to 1000 ml.

### Preparation of Stock Solutions

To make 1M MgSO₄: 120.37 g dissolved in 1000 ml DI water. Sterilized by autoclaving.

To make 1M CaCl₂: 147.01 g g dissolved in 1000 ml DI water. Sterilized by autoclaving.

To make 1M glucose stock solution: 900 mL DI water, 180.16 g glucose, and complete to 1000 ml.

To make 200 g/L (20%) glucose stock solution: 900 ml DI water, 200 g glucose, and complete to 1000 ml. Filter sterilized by passing it thought a 0.22 μm filter and stored at 4C.

To make 1M CaCl₂-2H₂O: 15 ml DI water, 2.94 g CaCl₂-2H₂O, and complete to 20 ml.

To make 0.1M MnCl₂-4H₂O: 7 ml DI water, 0.1979 g MnCl₂-4H₂O, and complete to 10 ml.

To make 0.1 M CuCl₂-2H₂O: 7 ml DI water, 0.1705 g CaCl₂-2H₂O, and complete to 10 ml.

To make 0.1 M MgSO₄: 17 ml DI water, 0.2407 g Mg2SO₄, and complete to 20 ml.

To make 5×M9 salts (per liter): 64 g Na₂HPO₄.7H₂O, 15 g KH₂PO₄, 2.5 g NaCl, 5.0 g NH4Cl. Sterilized by autoclaving for 25 minutes at 15 psi on the liquid cycle.

TABLE 2
Micronutrients Solution for 1 L of medium:

| | | | | | Final concentration | | |
|---|---|---|---|---|---|---|---|
| 39.4 | μL | CaCl₂—2H₂O | 1 | M | Ca | 1.576 | mg/L |
| 2.185 | μL | MnCl₂—4H₂O | 0.1 | M | Mn | 0.012 | mg/L |
| 81.9 | μL | MgSO₄ | 0.1 | M | Mg | 0.199 | mg/L |

TABLE 3
1000× Trace Metals Stock Solution
prepared as described in (Studier 2005):

| | | | |
|---|---|---|---|
| 3.9 | ml | | dIH₂O |
| 200 | μL | 1M | CaCl₂ |
| 200 | μL | 0.1M | CuCl₂—6H₂O |
| 200 | μL | 0.1M | Na₂MoO4—2H₂O |
| 200 | μL | 0.1M | H₃BO₃ |
| 100 | μL | 1M | MnCl₂—4H₂O |
| 100 | μL | 1M | ZnSO₄—7H₂O |
| 100 | μL | 0.2M | NiCl₂—6H₂O |

### Results

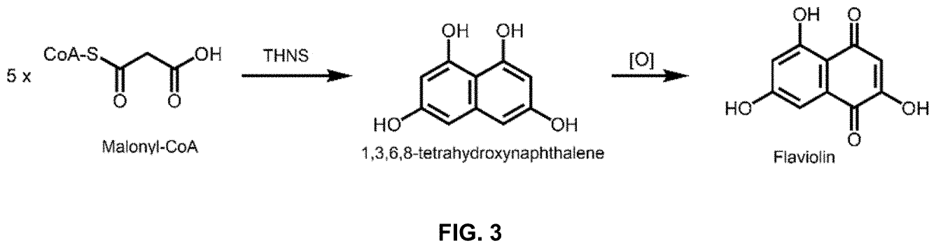

The in vivo assay for ACC is based on the enzyme 1,3,6,8-tetrahydroxynapthalene synthase (THNS). The only substrate for THNS is malonyl-CoA, and the product is 1,3,6,8-tetrahydroxynapthalene (THN). Under aerobic conditions, THN undergoes spontaneous oxidation to form flaviolin, which is secreted out of the cell (Izumikawa, et al. 2003. J Ind Microbiol Biotechnol 30(8):510-15). Flaviolin can be quantitated spectrophotometrically. Since ACC is the only enzyme in E coli that produces malonyl-CoA, THNS is specific for measuring activity of ACC in vivo. The concentration of intracellular malonyl-CoA can be directly calculated as five times the achieved concentration of flaviolin (FIG. 3).

When E. coli cultures are grown to produce large quantities of native or heterologous proteins, the cost of protein production is important. It is therefore advantageous to grow cells in the medium that achieves the highest culture yield for a given amount of carbon source and nutrients.

Growth in Luria-Bertani (LB) broth is carbon limited, indicating that LB broth contains <100 μM fermentable sugar equivalents utilizable by E. coli (free sugars, sugar phosphates, oligosaccharides, nucleotides, etc.). Since LB broth lacks recoverable sugars and has high concentrations of catabolizable aminoacids, probably these are depleted sequentially during the post exponential phase of growth. This phenomenon causes a constant variation in the physiological state of the cells. Furthermore, commercial sources of LB were observed to vary from batch to batch, introducing further variability into the system.

This condition of instability was seen many times in the data results, in which the reproducibility was difficult to reach when the experiment was repeated many times with the same setting, indicating that the system is very sensitive, and that an accurate and careful control is required to guarantee an optimized system. In that way, several variables were studied to overcome this issue. Specifically, the time of incubation of the inoculum, incubation temperature, trace metals presence in the medium, carbon source availability in the inoculum, type of carbon source, minimal and rich medium, pH of the inoculum, aeration, amount and type of inducer for gene expression, time of induction and temperature shift, were tested to develop a tight method in which all conditions are well known and established.

### Rich Medium

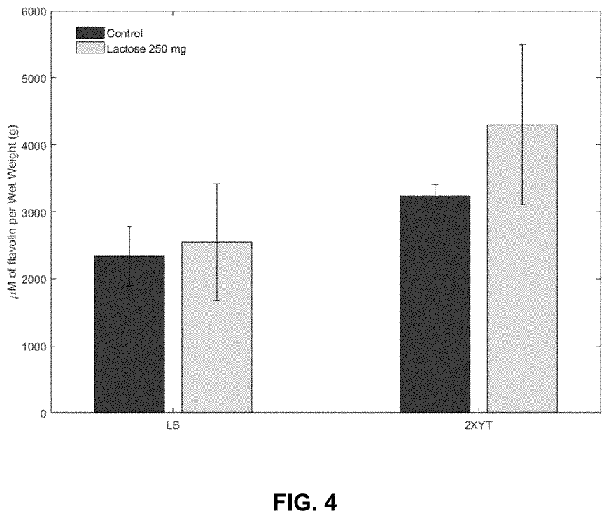

E. coli cells were grown in a LBT or 2XYT 10 ml inoculum with antibiotic where appropriate. The inoculums were incubated aerobically for 24 hours at 37° C. Cultures were grown in triplicate in 5 mL LB and a 1% (v/v) inoculum was introduced. No carbon supplementation was done during any step. Induction was done with the addition of 250 mg of lactose. After 24 hours of incubation of the cultures, the flaviolin absorbance was measured (FIG. 4). The basic components in LB and 2XYT medium are tryptone, yeast extract, and sodium chloride. However, 2XYT has 50% more yeast extract and 62.5% more tryptone than the LB, which suggested the lack of a carbon source in the inoculum, was the main variable to result in the different flaviolin production.

### Carbon Supplementation

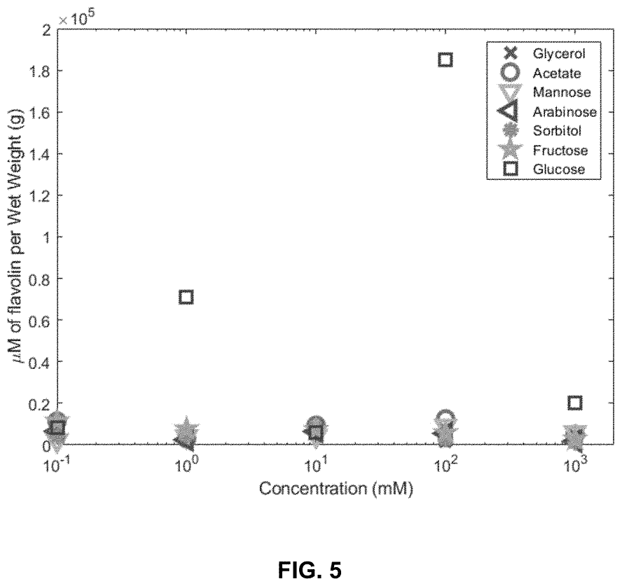

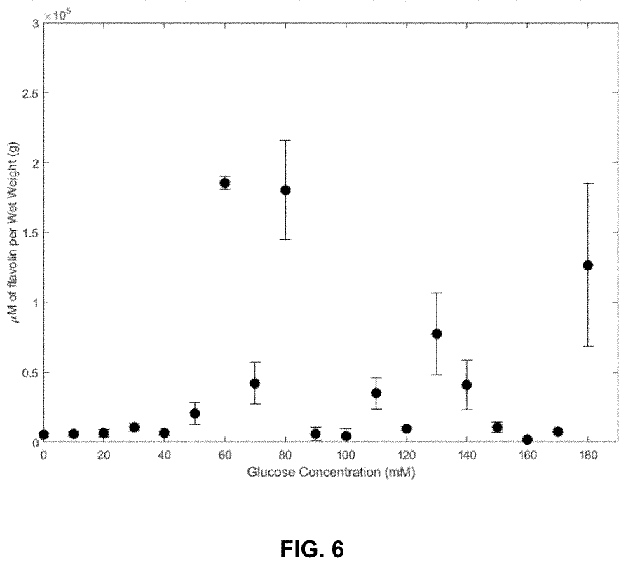

The effect on flaviolin production of preparing the inoculum with LBT medium supplemented with different carbon sources is shown in FIG. 5. Bacterial cultures that were inoculated with cells grown in LBT medium supplemented with 100 mM glucose produced the highest level of flaviolin. A wide range of concentrations (0.1 mM to 1 M) was initially examined, however, a more narrow range of glucose concentrations showed resulted in the highest level of flaviolin production and reproducibility (FIG. 6).

### Minimal Medium

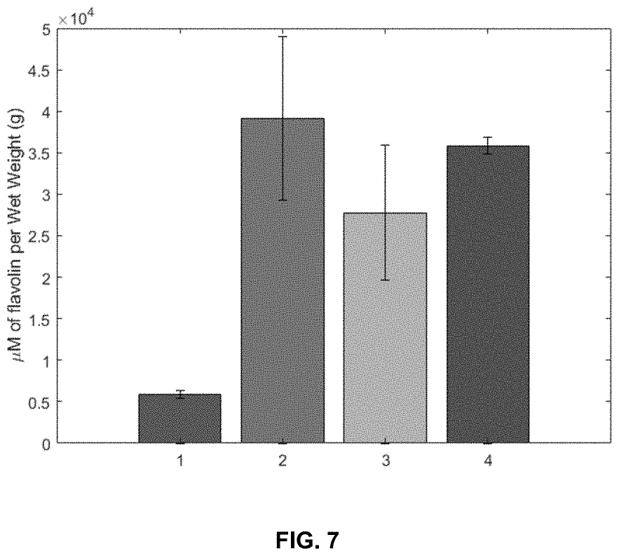

The ability to utilize minimal media in bioprocess engineering would be advantageous in terms of low cost and reproducibility because every component is controlled by the investigator. Therefore, the effect of the minimal medium M9 on flaviolin production was examined. The inoculum and cultures that were tested are shown in Table 4. The production in flaviolin in a minimal medium was statistically the same as growth in LBT medium (FIG. 7).

TABLE 4
Medium utilized to prepare inoculum and cultures

| Sample | Inoculum | Cultures |
|---|---|---|
| 1 | LBT | LBT |
| 2 | LBT 150 mM Glu | LBT |
| 3 | LBT | M9 |
| 4 | LBT 150 mM Glu | M9 |

### Richer Medium Supplemented With Glucose

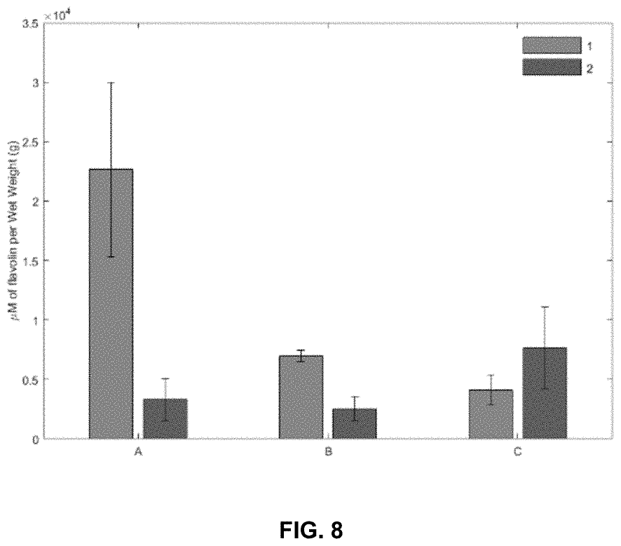

A more rich medium than 2XYT—Terrific Broth—was tested for both the inoculum and the culture medium. Terrific Broth contains 41.7% more yeast extract than 2XYT and also contains glycerol. When Terrific Broth was used for either the inoculum or culture medium or both the level of flaviolin production was significantly lower than when LB medium supplemented with 80 mM glucose was used as the inoculum and LB medium was used as the culture medium (FIG. 8).

TABLE 5
Medium utilized to prepare inoculum and cultures

| Sample | Inoculum | Cultures |
|---|---|---|
| A1 | LBT 80 mM Glu | LB |
| A2 | LBT 80 mM Glu | TB |
| B1 | TB | LB |
| B2 | TB | TB |
| C1 | TB 80 mM Glu | LB |
| C2 | TB 80 mM Glu | TB |

### Effect of Metal Ions on Flaviolin Production

The evaluation of different carbon sources on flaviolin production led to an unexpected finding. Namely, the reproducibility of flaviolin production depended on the type of water used to prepare the medium. Bacterial cells produced flaviolin much more reliably when tap water was used to prepare the medium compared to distilled water. This led to the hypothesis that metal ions in the tap water maybe effecting flaviolin production. An elemental analysis of the tap water revealed significant concentrations of: aluminum (0.035mg/L), calcium (1.58mg/L), magnesium (0.199mg/L), and manganese (0.012 mg/L).

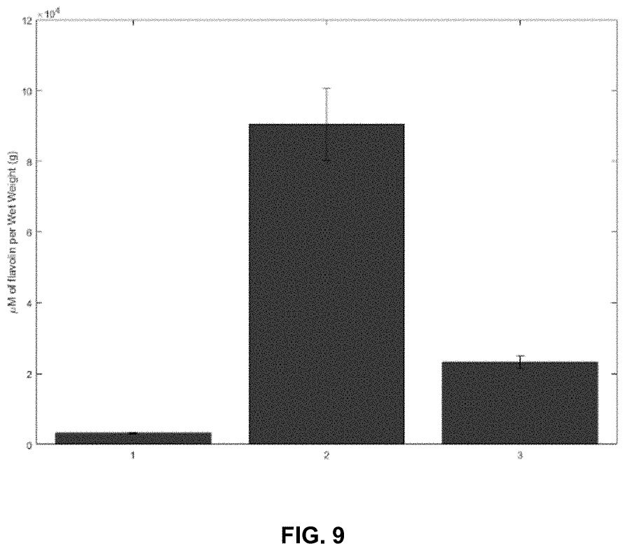

To test if these metal ions affected flaviolin production various combinations of the above metals are added to the medium prepared with distilled water (Table 6). Not surprisingly, aluminum was found to have no effect on flaviolin production. In contrast, calcium, magnesium and manganese when added together had the most significant effect on the amount and, most importantly, reproducibility of flaviolin production (FIG. 9). It is not surprising these metals have a significant effect since these metals act as cofactors for enzymes involved in vital metabolic processes such as DNA replication, transcription and translation.

TABLE 6
Supplementation of metals into the
medium for inoculums and cultures

| Sample | Metal Concentration in the Medium |
|---|---|
| 1 | — |
| 2 | 1.576 mg/L Ca, 0.012 mg/L Mn, 0.199 mg/L Mg |
| 3 | 4.274 mg/L Ca, 0.032 mg/L Mn, 0.063 mg/L Cu, |
| | 0.309 mg/L Mg |

### Induction: IPTG or Lactose

If biochemical engineering is going to be a viable alternative to fossil fuels for production of industrial chemicals it must be economically competitive. The standard procedure for inducing expression of genes controlled by the lac operon is to add the gratuitous inducer Isopropyl β-D-1-thiogalactopyranoside (IPTG). The cost of IPTG is $65.00/g. In contrast, the cost of lactose, the natural inducer of the lac operon, is $0.05/g. Therefore, lactose was tested as an alternative for IPTG for induction of the lac operon. The inoculum was prepared using LBM supplemented with 60 mM of glucose and incubated for 22 hours at 37 ° C. Cultures were grown in triplicate in LBM, and a 1% (v/v) inoculum was inoculated. The inducer was added at the time of the inoculation. After 24 hours of incubation of the cultures, the flaviolin absorbance was measured

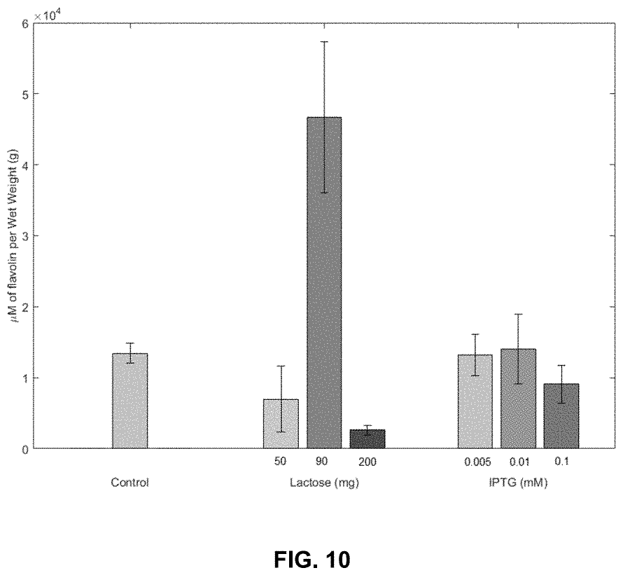

As shown in FIG. 10, lactose was far superior to IPTG in its ability to induce gene expression from the lac operon. Natural sugar inducers, such as lactose, have been shown to cause less stress and toxicity than IPTG in E. coli BL21(DE3) strains.

It was seen that heterologous proteins expressed at high levels in E. coli often fail to reach their native conformation and have a tendency to form inclusion bodies, but this can be minimized by culturing cells at a reduced growth temperature (Gadgil, et al. 2005. Biotechnology Progress 21(3):689-99). Also, plasmid stability can be improved when the induction phase is carried out at low temperatures (Zhang, et al. 2003. Protein Expression and Purification 29(1):132-39). Furthermore, once lac operon can be inhibited by glucose, the ideal time to induct the culture is when glucose is nearly exhausted. Usually, this occurs when OD₆₀₀ reaches 0.3, which in our case corresponds to roughly 2 hours of incubation.

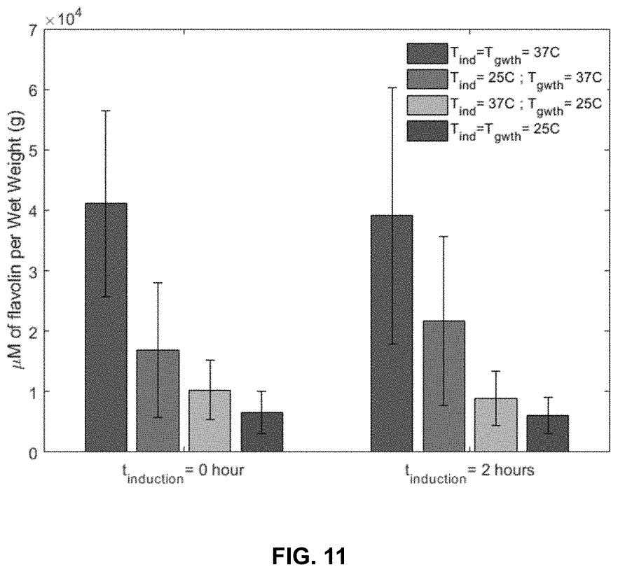

Hence, further experimentation was done to determine temperature dependence, and time of induction to verify the optimal condition to overexpress the target protein. With an 80mM glucose supplemented LBT inoculum incubated for 24 hours at 37° C., four cases of temperature shift were performed. Cultures were grown in triplicate in 5 ml of LBT medium without carbon supplementation and a 1% (v/v) inoculum was introduced. The time of induction was also evaluated, in which half of the samples were induced with 100 mg of lactose just after inoculating the inoculum and the other half of the samples was induced when OD₆₀₀=0.3 (2 hours of growth) was reached. After 24 hours of incubation of the cultures, the flaviolin absorbance was measured (FIG. 11).

### Inoculum Incubation Time and Temperature

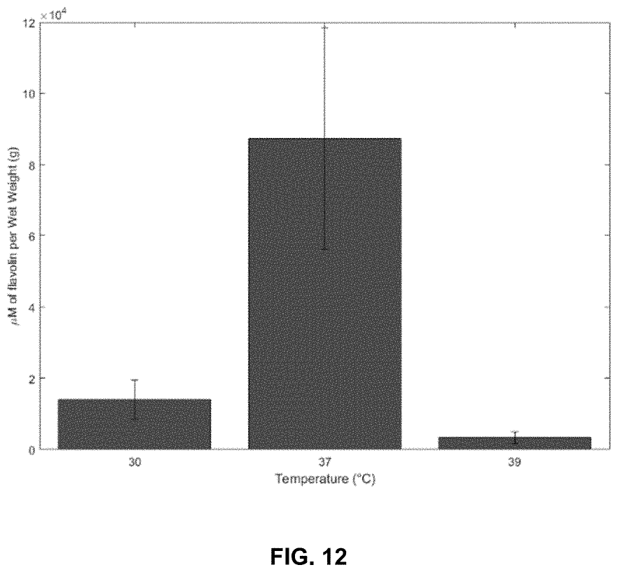

It is known that some metabolic pathways are activated in specific ranges of temperature. For this reason, it was tested the impact of the temperature during the inoculum phase by cultivating samples at different temperatures. E. coli cells were grown for 22 hours at 30, 37 and 39° C. in inoculums made with LBM medium supplemented with 60 mM of glucose. Cultures were grown in triplicate in 5 ml of LBM medium without carbon supplementation and a 1% (v/v) inoculum was introduced. Induction was done with the addition of 90 mg of lactose. After 24 hours of incubation of the cultures, the flaviolin absorbance was measured (FIG. 12).

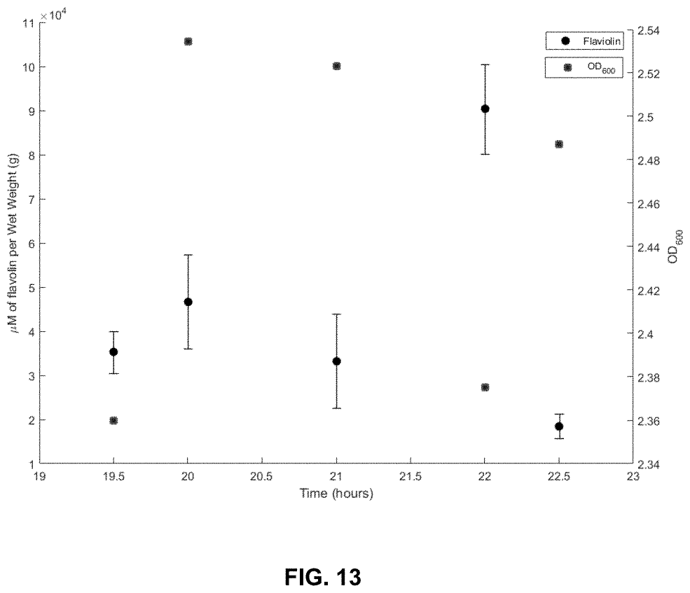

Standard microbiology procedure indicates “overnight” as the period to incubate the inoculum without specifying the OD₆₀₀. Therefore, with the aim of quantifying how many hours the inoculum needed to achieve an optimal high cell concentration to overexpress THNS during induction phase, an experiment varying the time for incubation of the inoculum was done. E. coli cells were grown at 37° C. in inoculums made with LBM medium supplemented with 60 mM of glucose. Inoculums were incubated for 19.5, 20, 20.5, 21, 21.5, 22 and 22.5 hours, and the respective optical density at 600 nm was measured. Cultures were grown in triplicate in 5 ml of LBM medium without carbon supplementation and a 1% (v/v) inoculum was introduced. Induction was done with the addition of 90 mg of lactose. After 24 hours of incubation of the cultures, the flaviolin absorbance was measured (FIG. 13).

### Aeration

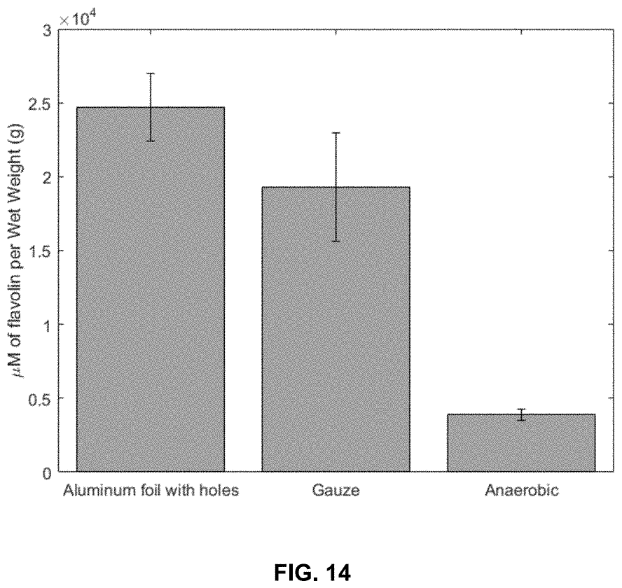

It is known that keeping a reasonably good aeration is essential to maintain a neutral pH and obtain a good cellular growth. Furthermore, the biosynthesis of microbial products in shake flasks may be limited by inadequate supply of oxygen to the cultures (McDaniel, et al. 1969. Applied Microbiology 17(2):286-90). Therefore, the influence of three types of closure was tested in order to understand the role of oxygen supply in the production of malonyl-CoA. E. coli cells were grown for 22 hours at 37° C. in inoculums made with LBM medium supplemented with 60 mM of glucose. Cultures were grown in triplicate in 5 ml of LBM medium without carbon supplementation and a 1% (v/v) inoculum was introduced. Induction was done with the addition of 90 mg of lactose. Flasks utilized to make either the inoculum or the cultures were covered with aluminum foil with holes, cotton gauze, or with parafilm. Three layers of parafilm sealed completely the exchange of air leading to an anaerobic environment inside the flask. After 24 hours of incubation of the cultures, the flaviolin absorbance was measured (FIG. 14).

### Overexpressing ACC

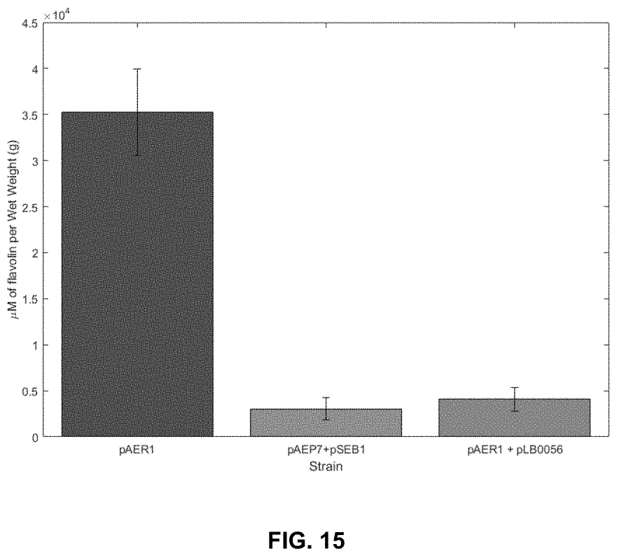

Since the initial objective of optimizing ACC activity in vivo is to increase production of malonyl-CoA, the original hypothesis was that this could be accomplished by overproduction of holo ACC. Therefore, the effect of overexpressing holo ACC genes on the amount of flaviolin produced was investigated. The strain consisted of pAEP7 and pSEB1, which together contained the genes for THNS and all three ACC subunits (one gene coding for BCCP and BC; two genes coding for CT) (Table 1). Additionally, in order to provide enough biotin ligase which is responsible for the biotinylation of BCCP, another strain containing pAER1 and pLB0056, which coded for the genes of all three ACC subunits as well as for biotin ligase, was tested. Therefore three strains of E coli (pAER1, pAEP7+pSEB1, pAER1+pLB0056) were grown for 19.5 hours at 37 ° C. in inoculums made with LBM medium supplemented with 60 mM of glucose. Cultures were grown in triplicate in 5 ml of LBM medium without carbon supplementation and a 1% (v/v) inoculum was introduced. Induction was done with the addition of 90 mg of lactose. After 24 hours of incubation of the cultures, the flaviolin absorbance was measured (FIG. 15).

### Effect of CO₂

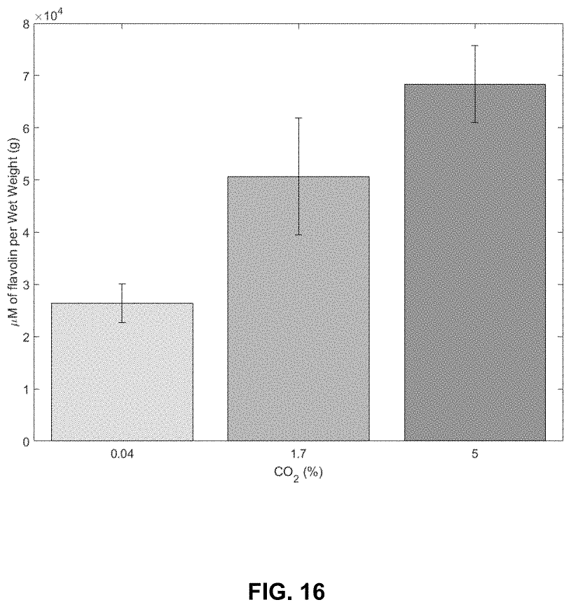

Another fundamental tenet of enzyme kinetics is that if the concentration of substrate is increased the reaction velocity will increase. One of the substrates of ACC is bicarbonate. Since the level of CO₂ in the air remains relatively constant the concentration is controlled by the intracellular pH. At an intracellular pH of 7.0 equilibrated with air (330 ppm CO₂) the level of bicarbonate is 50.1 μM, which is well below the Km value (0.37 mM) for bicarbonate in E. coli biotin carboxylase (Asada. 1982. John Wiley & Sons). Therefore, the effect of increased levels of CO₂ on flaviolin production were investigated. Bacteria were cultured in an environment of 5% CO₂ and compared to bacteria cultured in an environment of air (0.04% CO₂). As can be seen in FIG. 16, the flaviolin production in bacteria cultured in 5% CO₂ was 2.5 times greater than flaviolin production in bacteria cultured in air. The effect of CO₂ on flaviolin production is dose-dependent because when bacteria were cultured in 1.7% CO₂ there was 1.5 fold lower than the value at 5% CO₂. Thus, the fact that CO₂ has such a pronounced positive effect on flaviolin production, and by inference malonyl-CoA, not only makes the methodology in this invention have a lower carbon footprint but it also means one of the most important nutrients is overly abundant.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

What is claimed is:

1. A method for maximizing malonyl-CoA production in bacterial culture, comprising
(a) preparing a bacterial inoculum by culturing a bacterial colony in a bacterial medium at from 30 to 39° C. for from 12 to 48 hours under aerobic conditions,
wherein the bacterial medium is produced from purified water supplemented with from 0 to 0.5 mg/L magnesium, from 0 to 0.1 mg/L manganese, from 0 to 6 mg/L calcium, or any combination thereof,
wherein the bacterial medium comprises from 5 to 1000 mM glucose, and
wherein the bacterial medium comprises a pH of about 6.0 to 7.5; and
(b) culturing the inoculum at from 0.5 to 2% (v/v) in a bacterial medium,
wherein the bacterial medium is produced from purified water supplemented with 0 to 0.5 mg/L magnesium, 0 to 0.1 mg/L manganese, 0 to 6 mg/L calcium, or any combination thereof, and
wherein the bacterial medium comprises from 0 to 35 mg/ml lactose at about 30-39° C. for about 24 hours under aerobic conditions.

2. The method of claim 1, wherein the aerobic conditions comprise from 0.04 to 25% concentration of CO₂ and 0% to 25% concentration of O₂.

3. The method of any one of claims 1, wherein the bacterial medium comprises LB, TB, 2XYT, or any combination of yeast extract and tryptone, and MOPS, or M9 minimal medium.

4. The method of claim 1, wherein the bacterial colony comprises E. coll.

5. The method of claim 1, wherein the bacterial medium of step (b) does not comprise IPTG.

6. The method of claim 1, wherein the bacterial medium of step (b) does not comprise glucose.

7. The method of claim 1, further comprising product extraction.

8. The method of claim 7, wherein the final product is secreted out of the cell and the cells are discarded.

9. The method of claim 7, wherein the product remains in the cytosol and the cells are frozen.

10. The method of claim 1, wherein the bacterial colony is recombinantly engineered to overexpress malonyl-CoA.
